(19) United States
(12) Patent Application Publication
(10) Pub. No.: US 2010/0217008 A1
(43) Pub. Date: Aug. 26, 2010

(54) PROCESS FOR THE PREPARATION OF VALSARTAN AND INTERMEDIATE PRODUCTS

(75) Inventors: Jiri Stohandl, Bobrova (CZ); Pavel Bobal, Malacky (SK); Jaroslav Frantisek, Brno (CZ)

Correspondence Address: SMITH PATENT CONSULTING, LLC, 515 East Braddock Road, Suite B, ALEXANDRIA, VA, 22314, US

(73) Assignee: ratiopharm GmbH, Ulm (DE)

(21) Appl. No.: 12/516,906

(22) PCT Filed: Dec. 11, 2007
(86) PCT No.: PCT/EP2007/010834

§ 371 Date: May 29, 2009

(30) Foreign Application Priority Data
Dec. 14, 2006 (EP) 06025988.4

(51) Int. Cl. C07D 413/04; C07D 333/22; C07D 257/04

(52) U.S. Cl. 548/237; 549/72; 548/253; 548/215

(57) ABSTRACT

The present invention relates to a new method for the production of valsartan, a valine derivative having the chemical N name is (S)—N-(1-carboxy-2-methylprop-1-yl)-N-pentanoyl-N-[2′-(1H-tetrazol-5-yl)-biphenyl-4-ylmethyl]amine, and pharmacologically acceptable salts thereof. Furthermore the invention relates to new intermediate compounds which are suitable for the production of valsartan and new methods for the production of intermediate compounds which are suitable for the production of valsartan.

The present invention relates to a new method for the production of valsartan, a valine derivative having the chemical name (S)—N-(1-carboxy-2-methylprop-1-yl-N-pentanoyl-N-[2′41H-tetrazol-5-yl)-biphenyl-4-ylmethyl]amine, and pharmacologically acceptable salts thereof. Furthermore the invention relates to new intermediate compounds which are suitable for the production of valsartan and new methods for the production of intermediate compounds which are suitable for the production of valsartan.

Valsartan and efficient and economic methods for its production are of considerable interest. It is a angiotensin II receptor antagonist and has proven to be a potent active agent for controlling high blood pressure in mammals including humans and secondary diseases arising there from.

Valsartan and its production have been described for the first time in EP-A-443983. The disclosed synthetic pathways comprises various steps among which oily intermediates are formed. Furthermore the synthesis comprises as an essential step an N-alkylation, the reaction of a primary amine with for instance a bromo methyl biphenyl derivative.

However, the above-mentioned synthetic processes still need to be improved in order to produce valsartan on an industrial scale. Intermediates as oily and/or high viscous liquids are very difficult to handle, to weight and to dry. Furthermore the total yield is still unsatisfactory.

Common to all synthesis routes is that a valine ester is prepared first and is then alkylated at the amine moiety. In this reaction, however, there is the possibility that an undesired double-alkylation occurs, forming not a secondary amine but a tertiary amine instead.

It is therefore an object of the invention to provide new synthetic processes and intermediate products for the production of valsartan and of its pharmacologically acceptable salts. In particular, it is an object of the invention to provide new synthetic processes and intermediate products for the production of valsartan and of its pharmacologically acceptable salts by which valsartan is obtainable in a high overall yield.

It is furthermore an object of the invention to provide new synthetic processes and intermediate products for the production of valsartan and of its pharmacologically acceptable salts, which can be produced using industrially readily obtainable starting materials and to avoid the use of toxic substances or substances for which there is a labeling obligation.

Accordingly the subject matter described above has been found.

A key aspect of the invention is the production of a compound of general formula I

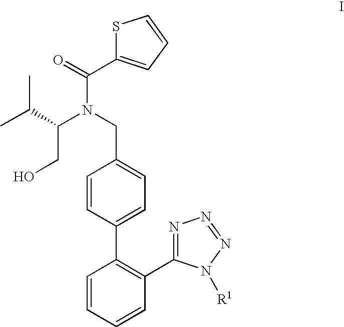

wherein R¹ represents hydrogen or a tetrazole protecting group.

Suitable tetrazole protecting groups in the residue of the above-given general formula I are known from EP-A-291969. Suitable tetrazole protecting groups are in particular triphenylmethyl, 1-methyl-1-phenylethyl or tert-butyl.

The compounds of general formula I are prepared by reacting a compound of general formula II

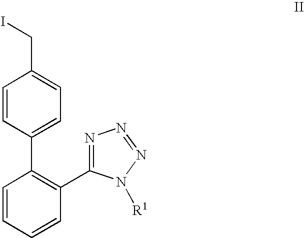

wherein R¹ represents hydrogen or a tetrazole protecting group and L represents a leaving group, such as halogen or an other suitable leaving group, preferably selected from the group consisting of Cl, Br, I, triflate, mesylate, tosylate, most preferably Br with a compound of general formula III or a pharmaceutically acceptable salt thereof

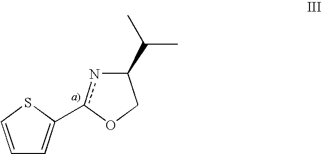

wherein a) denotes a double bond or a single bond and wherein when a) denotes a single bond the nitrogen atom is additionally substituted by a hydrogen atom.

Thus, the compound of general formula III is selected from the group consisting of a compound of general formula IIIa

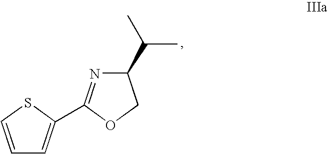

a compound of general formula IIIb

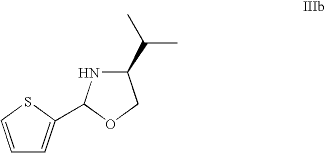

and pharmaceutically acceptable salts thereof.

The above described reaction is preferably carried out in the presence of a Bronsted base. Examples of suitable Bronsted bases are alkali metal carbonates or alkali metal bicarbonates, such as, for example, sodium carbonate, potassium carbonate or sodium bicarbonate. Potassium carbonate is preferred.

The above described reaction is advantageously carried out in the presence of an activator. The activator activates the reaction of a compound of general formula II with a compound of general formula III to give a compound of general formula I. Examples of suitable activators are alkali metal halides or earth alkali metal halides, such as, for example, alkali metal chlorides, alkali metal bromides, alkali metal iodides, earth alkali metal chlorides, earth alkali metal bromides or earth alkali metal iodides, preferably alkali metal iodides, such as potassium iodide or sodium iodide. Potassium iodide is especially preferred.

The reaction is carried out in a suitable inert solvent. Examples of such suitable inert solvents are ethers (preferably diethyl ether), cyclic ethers (preferably tetrahydrofuran), ketones (preferably acetone, methylisobutylketone or methylethylketone), chlorinated hydrocarbons (preferably dichloromethane) or nitrogen containing organic solvents (preferably N-methylpyrrolidone). Methylethylketone is especially preferred.

The reaction is preferably carried out at elevated temperatures, preferably at temperatures between 50° C. and the boiling point of the solvent.

The reaction of a compound of general formula II with a compound of general formula IIIb to give a compound of general formula I is carried out as a two step process. In a first step a compound of general formula II is reacted with a compound of general formula IIIb, following the procedure given above. Optionally the reaction product of the reaction of a compound of general formula II with a compound of general formula IIIb can be isolated. In a second step the reaction product of the reaction of a compound of general formula II with a compound of general formula IIIb is converted into a compound of general formula I by an oxidative ring opening reaction.

The oxidative ring opening reaction is preferably carried out using a suitable oxidant. Examples of such suitable oxidants are N-halosuccinimides like N-bromosuccinimide (NBS) or N-chlorosuccinimide, halides like chlorine, bromine or iodine, peroxides like ozone, H₂O₂, KMnO₄, K₂Cr₂O₇, NalO₄ or trifluoroperoxyacetic acid, hypohalides like hypochloride, hypobromide or hypoiodide, or bromates, chlorates, perchlorates and perbromates. N-halosuccinimides and hypohalides are preferred. NBS and hypochlorides are especially preferred.

The oxidative ring opening reaction is advantageously carried out in the presence of a Bronsted base. Suitable Bronsted bases are alkali metal carbonates, alkali metal bicarbonates or alkali metal hydroxides, such as, for example, sodium hydroxide, potassium hydroxide, sodium carbonate, potassium carbonate or sodium bicarbonate. Potassium carbonate is preferred.

If a compound of general formula II wherein R¹ represents a tetrazole protecting group is used in the reaction of a compound of general formula II with a compound of general formula IIIb, the tetrazole protecting group of the reaction product can, if desired, be removed prior to or after the oxidative ring opening reaction by the reaction with a suitable acid.

Examples of suitable acids are Lewis acids like metal halides, such as metal chlorides, metal bromides or metal iodides, or Bronsted acids like strong organic or inorganic acids, such as hydrochloric acid, hydrobromic acid, sulfuric acid, trichloroacetic acid, trifluoroacetic acid or methanesulfonic acid. Preferably zinc halides or strong organic acids are used. Most preferably the reaction is carried out with zinc chloride or methanesulfonic acid.

Compounds of the general formula I wherein R¹ denotes a tetrazole protecting group can be converted into compounds of general formula I wherein R¹ denotes hydrogen by the reaction of compound of general formula I wherein R¹ denotes a tetrazole protecting group with a suitable Lewis acid to give a compound of general formula I wherein R¹ denotes hydrogen.

Examples of suitable Lewis acids are metal halides, such as metal chlorides, metal bromides or metal iodides. Preferably zinc halides are used. Most preferably reaction is carried out with zinc chloride.

By the above described method compounds of general formula I can be obtained in high yield and high purity. Furthermore no racemisation occurs so that compounds of general formula I in high enantiomeric purity can be obtained. Compounds of general formula I in solid state are well crystalline materials which can be handled easily (e.g. separated, filtered, purified, dried, weighted, transported and/or stored).

The production of compounds of the general formula II is known from EP-A-291969.

In general, the production of compounds of the general formula III wherein a) denotes a double bond (i.e. compounds of the general formula IIIa) is effected by reacting a compound of general formula IV

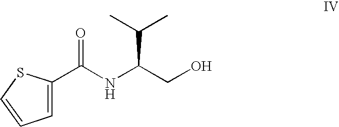

with a suitable cyclisizing agent. Preferably such cyclisizing agents have dehydrating properties. Thionyl chloride is especially preferred. Optionally a dehydrating agent can be added, such as molecular sieves or a Dean-Stark apparatus can be used.

The reaction is usually carried out in a suitable inert solvent, e.g. aliphatic and aromatic hydrocarbons such as hexanes, benzene, toluene, xylenes or mixtures thereof at temperatures between room temperature and the boiling point of the suitable inert solvent. Most preferably reaction is carried out using the cyclisizing agent as solvent.

The production of compounds of the general formula IV is effected by reacting a compound of general formula V

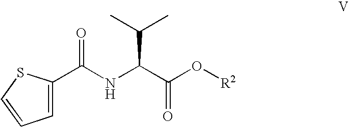

wherein R² represents alkyl or benzyl, especially C1 to C4 alkyl, preferably methyl, ethyl, propyl or butyl with methyl being especially preferred, with a suitable reducing agent. Preferably such reducing agents are hydrides. NaBH₄ is especially preferred.

The reaction is usually carried out in a suitable inert solvent, e.g. aliphatic alcohols such as methanol, ethanol, propanol, iso-propanol or in water or in mixtures of aliphatic alcohols and water. Most preferably reaction is carried out in water.

The above described reaction is advantageously carried out in the presence of an activator. The activator activates the reaction of a compound of general formula V with a suitable reducing agent to give a compound of general formula IV. Suitable activators are alkali metal halides or earth alkali metal halides, such as, for example, alkali metal chlorides, alkali metal bromides, alkali metal iodides, earth alkali metal chlorides, earth alkali metal bromides or earth alkali metal iodides, preferably earth alkali metal chlorides, such as calcium chloride or magnesium chloride. Calcium chloride is especially preferred.

The production of compounds of the general formula V is effected by reacting a compound of general formula VI

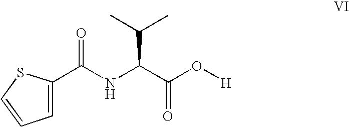

with a chlorinating agent and a compound of the general formula R²—OH.

The chlorinating agents are suitable to convert carboxylic acids into carboxylic acid chlorides. Sulphuryl chloride is especially preferred as chlorinating agent.

The reaction is usually carried out in a suitable inert solvent, e.g. aliphatic alcohols such as methanol, ethanol, propanol, iso-propanol at temperatures between −20° C. and 50° C. Preferably reaction is carried out in R²—OH at room temperature.

Most preferably the reaction of compound of the general formula VI with a chlorinating agent and with compound of the general formula R²—OH to give a compound of general formula V and the reaction of a compound of general formula V with a suitable reducing agent to produce a compound of the general formula IV is carried out as a one-pot reaction without the isolation of a compound of the general formula V.

The production of compounds of the general formula VI is effected by reacting a compound of general formula VII

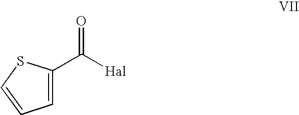

wherein Hal represents halogen preferably selected from the group consisting of Cl, Br, I, preferably Cl with a compound of general formula VIII

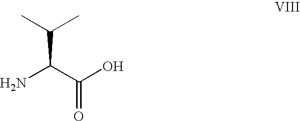

preferably in the presence of a Bronsted base. Suitable Bronsted bases are alkali metal carbonates, alkali metal bicarbonates or alkali metal hydroxides, such as, for example, sodium carbonate, potassium carbonate, sodium bicarbonate, sodium hydroxide or potassium hydroxide. Potassium hydroxide is preferred.

The preparation of compounds of the general formula III wherein a) denotes a single bond and wherein the nitrogen is additionally substituted by a hydrogen atom (i.e. of compounds of the general formula IIIb) is effected by reacting L-valinol with thiophene-2-carbaldehyde. The reaction is preferably carried out using water removal methods. The water removal methods can be for example azeothropic distillation, the use of a Dean-Stark apparatus or the addition of water absorbing agents like magnesium sulfate, sodium sulfate, phosphorous pentoxide, or molecular sieves like zeolites A, especially zeolite A3, A4 or A5. A zeolite molecular sieve of type A3 is preferred.

The preparation of valsartan is carried out by
an oxidation of a compound of general formula I with a suitable oxidizing agent to give a compound of general formula X,

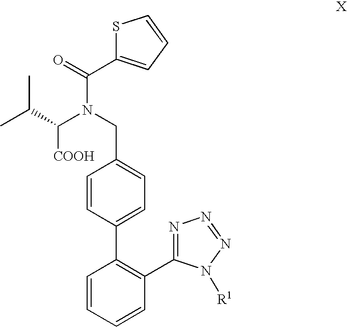

wherein R¹ represents hydrogen or a tetrazole protecting group and
conversion of a compound of general formula X to valsartan by reaction with a suitable hydrogenating agent.

Suitable oxidizing agents oxidize primary alcohols to carboxylic acids and can be for example N-halosuccinimides like N-bromosuccinimide (NBS) or N-chlorosuccinimide, halides like chlorine, bromine or iodine, peroxides (e.g. trifluoroperoxyacetic acid, NalO₄, potassium permanganate, hydrogen peroxide or benzoyl peroxide), ozone, chromium-(VI)-oxide, hypohalides like hypochloride, hypobromide or hypoiodide, or bromates, chlorates, perchlorates, perbromates and silver salts. Most preferably potassium permanganate is used.

Optionally the oxidation can be carried out as a two step oxidation with or without isolation of the reaction product of the first oxidation step. In the first step a compound of general formula I is oxidized using a suitable oxidizing agent to give a compound of general formula IX,

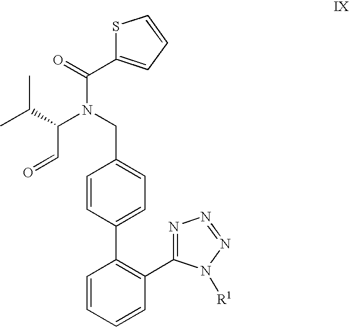

wherein R¹ represents hydrogen or a tetrazole protecting group.

Examples of suitable oxidizing agents are hypohalides like hypochloride, hypobromide or hypoiodide, or bromates, chlorates, perchlorates, perbromates and the swern oxidant (oxalyl chloride and DMSO) with hypochloride and the swern oxidant being preferred. Optionally an oxidation catalyst such as TEMPO (2,2,6,6-tetramethylpiperidine-1-oxyl) can be added.

In the second oxidation step a compound of general formula IX is oxidized using a suitable oxidizing agent to give a compound of general formula X. Preferably the suitable oxidizing agent is an oxidizing agent as defined above for the oxidation of primary alcohols to carboxylic acids.

An example of a suitable hydrogenating agent for the conversion of a compound of general formula X to valsartan is hydrogen, preferably in the presence of a metal catalyst such as nickel or palladium. Most preferably Raney nickel in the presence of methanol or water is used. The reaction can be carried out at normal pressure or preferably at elevated pressure.

Below the synthetic route is described and further explained showing one possible embodiment of the process of the present invention without intending to be limiting.

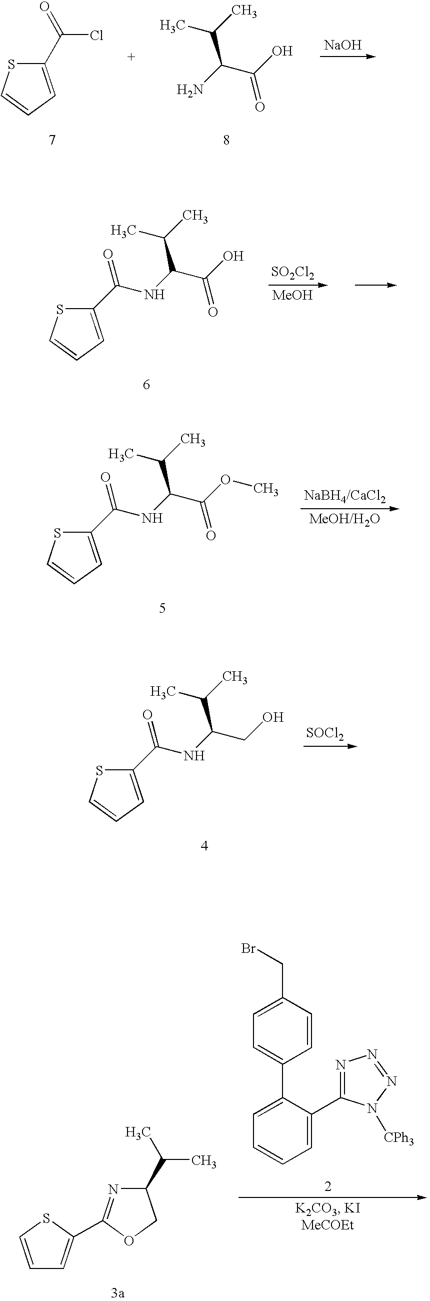

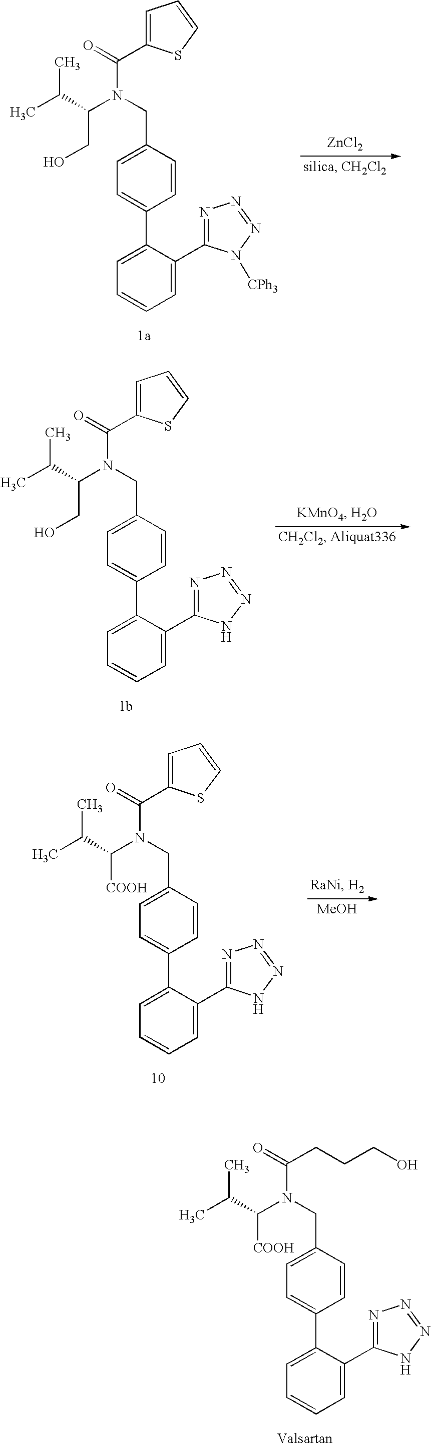

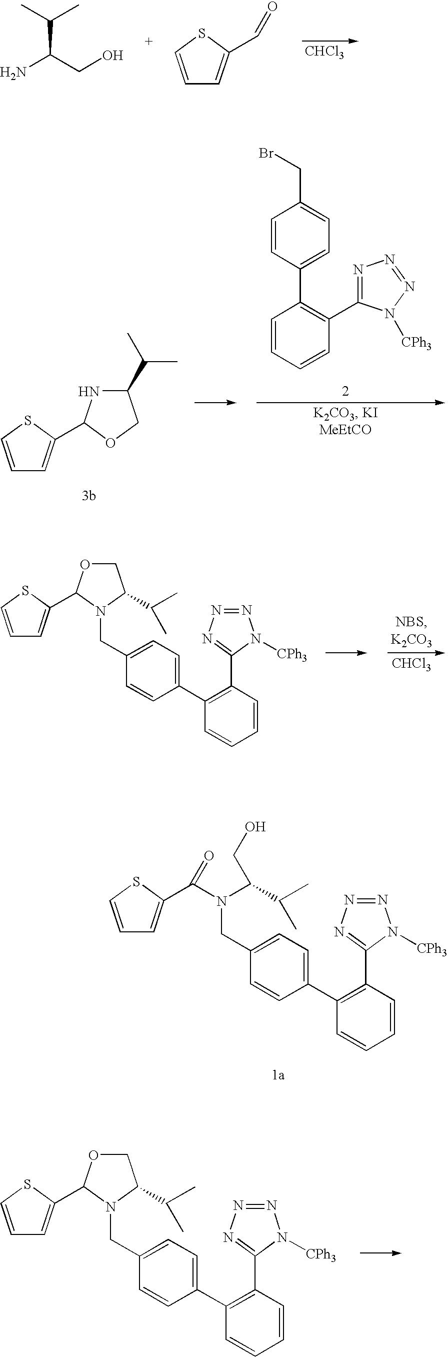

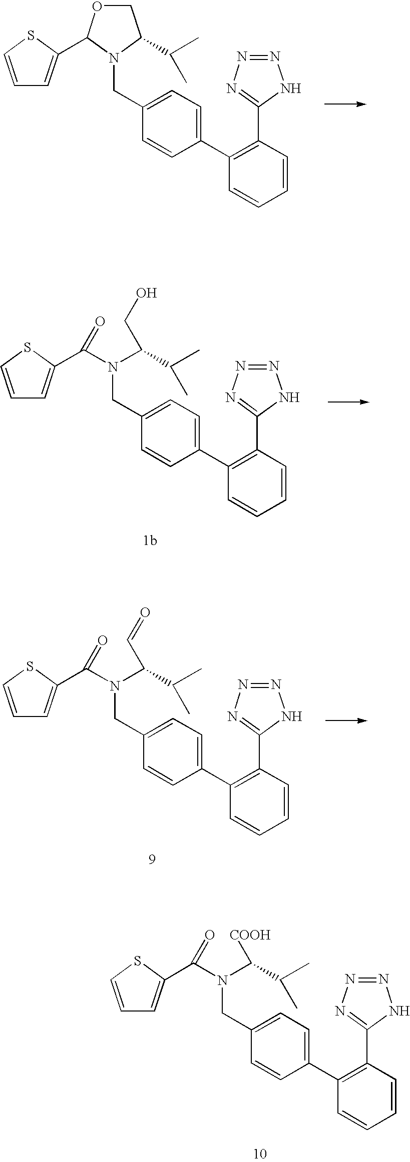

The compounds used and/or being formed in the respective synthetic routes are referred to by Arabic numerals. With respect to the compounds described in the reaction scheme, the following applies:
1a corresponds to a compound of general formula I with residue R¹ is CPh₃ (triphenylmethyl),
1b corresponds to a compound of general formula I with residue R¹ is hydrogen
2 corresponds to a compound of general formula II with residue R¹ is CPh₃ (triphenylmethyl) and Hal is chloride,
3a corresponds to a compound of general formula IIIa,
3b corresponds to a compound of general formula IIIb,
4 corresponds to a compound of general formula IV,
5 corresponds to a compound of general formula V with R² is methyl,
6 corresponds to a compound of general formula VI,
7 corresponds to a compound of general formula VII with Hal is chloride,
8 corresponds to a compound of general formula VIII,
9 corresponds to a compound of general formula IX with R¹ is hydrogen and
10 corresponds to a compound of general formula X with R¹ is hydrogen.

At the same time the compounds are preferred working examples of the compound groups defined by the respective general formulae.

### EXPERIMENTAL PART

All reactions were monitored by HPLC-PDA method and structures of all products, by-products and impurities were elucidated and confirmed by LC-MS-MS. Where the identification was not definite ¹H and ¹³C-NMR was used. The measured melting points are uncorrected.

### (2S)-3-methyl-2-((2thenoyl)-amino)butanoic acid (N-thenoyl-L-valine, 6)

L-valine (35.8 g, 0.306 mol) was dissolved in 2M NaOH (153 ml) and cooled down to 0° C. Thiopen-2-carbonyl chloride (44.9 g, 0.306 mol) and 4M NaOH (76.5 ml) was added drop wise alternately under stirring while maintaining the temperature between 0 and 5° C. Reaction mixture was let to warm up to room temperature within 20 minutes after the last addition. Reaction mixture was acidified with 32-% HCl to pH 2 and then extracted with ethylacetate (4-times 170 ml), dried over Na₂SO₄ and evaporated on RVE to yield 62.0 g (89%) of product. HR-MS: 228.0694 (MH⁺, calc. for C₁₀H₁₄NO₃S⁺: 228.0702).

### N-[(1S)-1-(hydroxymethyl)-2-methylpropyl]thiophene-2-carboxamide(N-thenoyl-L-valinol, 4)

N-thenoyl-L-valine (78.2 g, 0.344 mol) was dissolved in methanol (550 ml) and sulphuryl chloride (1.4 ml, 0.017 mol) was slowly added drop-wise. Reaction mixture was allowed to stand for 48 hours at laboratory temperature. Then methanol (1010 ml) and CaCl₂.2H₂O (152 g, 1.03 mol) dissolved in water (970 ml) were added. NaBH₄ (75.8 g, 2.003 mol) was added in small portions under cooling in the ice bath and stirring, and then left stand at laboratory temperature for 24 hours. White suspension was filtered off, washed with mixture of methanol-water 1:1 (twice 200 ml) and filtrate was concentrated in vacuo and extracted with chloroform (5-times 400 ml). Combined organic layers were re-extracted with brine, dried over Na₂SO₄ and evaporated on RVE to yield 62.5 g (85.2%) of white solid. HR-MS: 214.0902 (MH⁺, calc. for C₁₀H₁₆NO₂S⁺: 214.0900).

### (4S)-4-isopropyl-2-(2-thienyl)-4,5-dihydro-1,3-oxazole (3a)

N-thenoyl-L-valinol (20.0 g, 93.77 mmol) was added to thionyl chloride (20.0 ml, 275.7 mmol) and reaction mixture was stirred for 1 hour at laboratory temperature and evaporated on RVE to yield 20.0 g of the hydrochloride of title substance. The product was suspended in dichloromethane (200 ml) and 2M NaOH was added to adjust pH of aqueous layer to 9-10 (approx. 200 ml). Aqueous layer was separated and extracted with dichloromethane (twice 200 ml). Combined organic layer was dried over K₂CO₃ and evaporated on RVE to yield 16.7 g (91%) of free base. HR-MS: 196.0796 (MW, calc. for C₁₀H₁₄NOS⁺: 196.0791).

### (1S)—N-(1-Hydroxymethyl-2-methyl-propyl)-N′-[2′-(1-trityl-1H-tetrazol-5-yl)-biphenyl-4-ylmethyl]-thiophene-2-carboxamide (1a)

(4S)-4-isopropyl-2-(2-thienyl)-4,5-dihydro-1,3-oxazole (18.3 g, 93.71 mmol) was dissolved in 2-butanone (360 ml) and then 5-(4′-bromomethyl-biphenyl-2-yl)-1-trityl-1H-tetrazole (15.6 g, 93.98 mmol), KI (15.6 g, 93.98 mmol), K₂CO₃ (64.8 g, 486.9 mmol) and triphenylmethyl chloride (26.1 g, 91.61 mmol) were added. Reaction mixture was refluxed for 24 hours and then evaporated on RVE. Orange solid was dissolved in dichloromethane (300 ml) and extracted with saturated aqueous solution of Na₂S₂O₃ (300 ml) and water (twice 300 ml). Organic layer was dried over Na₂SO₄ and evaporated on RVE to yield 58.1 g (90%) of the product. It can be used without purification in the next step. HR-MS: 690.2903 (MH⁺, calc. for C₄₃H₄₀N₅O₂S⁺: 690.2902).

### (4S)-4-isopropyl-2-(2-thienyl)-1,3-oxazolidine (3b)

L-valinol (92.7 g, 0.9 mol) was dissolved in CHCl₃ (750 ml), and thiophene-2-carbaldehyde (100.8 g, 0.9 mol) and molecular sieve A3 (90 g) was added. The reaction mixture was refluxed for 2 hours. The molecular sieve was filtered off and the filtrate was evaporated on RVE yielding 158 g of the product. It can be used without purification in the next step. HR-MS: 198.0946 (MH⁺, calc. for C₁₀H₁₆NOS 198.0953)

### (1S)—N-(1-Hydroxymethyl-2-methyl-propyl)-N′-[2′-(1-trityl-1H-tetrazol-5-yl)-biphenyl-4-ylmethyl]-thiophene-2-carboxamide (1a)

(4S)-4-isopropyl-2-(2-thienyl)-1,3-oxazolidine (98.6 g, 0.5 mol) was dissolved in 2-butanone (750 ml), and 5-(4′-bromomethyl-biphenyl-2-yl)-1-trityl-1H-tetrazole (278.8 g, 0.5 mol), KI (83.0 g, 0.5 mol) and K₂CO₃ (69.0 g, 0.5 mol) were added. The reaction mixture was refluxed for 72 hours and the resulting yellow suspension was evaporated on RVE. The solid product was transferred to CHCl₃ (1.5 l) and extracted with water (1.5 l). The organic layer was washed thrice with 1.2 l of water and evaporated on RVE yielding 286.5 g (85%) of (4S)-3-[2′-(1-triphenylmethyl-1H-tetrazole-5-yl)-biphenyl-4-yl-methyl]-4-isopropyl-2-(2-thienyl)-1,3-oxazolidine (HR-MS: 674.2931 (MH⁺, calc. for C₄₃H₄₀N₅OS: 674.2954)). 29.6 g (44.0 mmol) of (4S)-3-[2′-(1-triphenylmethyl-1H-tetrazole-5-yl)-biphenyl-4-yl-methyl]-4-isopropyl-2-(2-thienyl)-1,3-oxazolidine were dissolved in CHCl₃ (100 ml). The solution was cooled to about 0° C., and K₂CO₃ (12.2 g, 88.0 mmol) and NBS (7.8 g, 44.0 mmol) were added. The reaction mixture was stirred for 1 hour at 0° C. and then extracted 6 times with 100 ml of water. The organic layer was dried over K₂CO₃ and evaporated on RVE to yield 20.6 g (68%) of the product. It can be used without purification in the next step.

### (1S)—N-(1-Hydroxymethyl-2-methyl-propyl)-N′-[2′-(1H-tetrazol-5-yl)-biphenyl-4-ylmethyl]-thiophene-2-carboxamide (1b)

(1S)—N-(1-hydroxymethyl-2-methyl-propyl)-N′-[2′-(1-trityl-1H-tetrazol-5-yl)-biphenyl-4-ylmethyl]-thiophene-2-carboxamide (64.6 g, 93.7 mmol) was dissolved in dichloromethane (650 ml) and silica gel (325 g) and ZnCl₂ (12.8 g, 93.9 mmol) was added. Reaction mixture was stirred for 1 hour at laboratory temperature and then filtered off. The silica gel was washed with dichloromethane (thrice 300 ml) and methanol (thrice 300 ml). Methanol fraction was evaporated on RVE to yield yellow amorphous product (41.9 g, 100%).

If desired the amorphous product can be further purified. It was dissolved in dichloromethane (1800 ml) and extracted with 0.4M NaOH solution (6-times 300 ml) and water (4-times 2000 ml). Combined alkaline and water layer were acidified with 35-% HCl to pH 3 and extracted with dichloromethane (thrice 400 ml). Organic layer was washed with water (twice 300 ml), dried over Na₂SO₄ and evaporated on RVE to yield 28.0 g (66.8%) of product. HR-MS: 448.1807 (MH⁺, calc. for C₂₄H₂₆N₅O₂S⁺: 448.1800).

### (4S)-3-[2′-(1H-tetrazole-5-yl)-biphenyl-4-yl-methyl]-4-isopropyl-2-(2-thienyl)-1,3-oxazolidine

(4S)-3-[2′-(1-triphenylmethyl-1H-tetrazole-5-yl)-biphenyl-4-yl-methyl]-4-isopropyl-2-(2-thienyl)-1,3-oxazolidine (20.0 g, 29.68 mmol) obtainable according to the previous example was suspended in methanol (130 mL) and 1.5 mL of methansulfonic acid were added drop wise during 30 minutes. The end of the reaction was indicated by dissolution of the suspension. The reaction mixture was stirred for additional 15 minutes and it was then checked for conversion. K₂CO₃ (5.6 g, 40.52 mmol) was added and it was stirred for 30 minutes. The reaction mixture was filtered and filtrate was evaporated on RVE to yield crystalline material. For further purification the crystals can be washed twice with diisopropyl ether (50 mL). The potassium salt of compound (4S)-3-[2′-(1H-tetrazole-5-yl)-biphenyl-4-yl-methyl]-4-isopropyl-2-(2-thienyl)-1,3-oxazolidine was obtained in nearly quantitative yield (10.2 g) and can be used directly in the next reaction step. HR-MS: 432.1859 (MH⁺, calc. for C₂₄H₂₆N₅OS: 432.1858).

### (2S)-3-Methyl-2-{N-[2′-(1H-tetrazol-5-yl)-biphenyl-4-ylmethyl]-N′-(thiophene-2-carbonyl)-amino}-butanoic acid (10)

Method A: (4S)-3-[2′-(1H-tetrazole-5-yl)-biphenyl-4-yl-methyl]-4-isopropyl-2-(2-thienyl)-1,3-oxazolidine (0.64 g, 1.48 mmol) was dissolved in chloroform (10 mL) and cooled down to 0° C. To the solution were consequently added KBr (0.18 g, 1.48 mmol), K₂CO₃ (0.41 g, 2.97 mmol) and KBF₄ (0.19 g, 1.48 mmol). A solution of 6-% NaOCl (1.52 mL, 1.48 mmol) in 8 mL of water was added drop wise at 0° C. The temperature of reaction mixture was let to increase to RT and then stirred for 1 hour. 2,2,6,6-tetramethylpiperidene-N-oxyl (2 mg, 0.015 mmol) and 6-% NaOCl (3.05 mL, 2.96 mmol) were added. The mixture was stirred until completion of the reaction. The water layer was neutralized with tartaric acid and separated and the organic layer was washed with 10 mL of 0.01M HCl and 10 mL of saturated solution of NaCl. Then the organic layer was extracted 6-times with 10 mL of 0.1M NaOH and then discarded. Water phases were carefully acidified with 0.1M HCl to pH 2 and extracted thrice with 20 mL of chloroform. Organic layer was dried over Na₂SO₄ and evaporated on RVE yielding 0.44 g (64%) of product. HR-MS: 462.1586 (MH⁺).

Method B: The reaction was done according to the method A with the addition of tetrabutylammonium bromide (0.05 g, 0.15 mmol).

Method C: (4S)-3-[2′-(1H-tetrazole-5-yl)-biphenyl-4-yl-methyl]-4-isopropyl-2-(2-thienyl)-1,3-oxazolidine (0.64 g, 1.48 mmol) was dissolved in acetonitrile (10 mL) and cooled down to 0° C. To the solution were consequently added KBr (0.18 g, 1.48 mmol) and K₂CO₃ (0.41 g, 2.97 mmol). A solution of 6-% NaOCl (1.52 mL, 1.48 mmol) was added drop wise during 15 minutes at 0° C. The temperature of reaction mixture was let to increase to RT and then stirred for 1 hour. 2,2,6,6-tetramethylpiperidene-N-oxyl (2 mg, 0.015 mmol) and 6-% NaOCl (3.05 mL, 2.96 mmol) were added. The mixture was stirred until completion of the reaction. The isolation of product was done according to the method A, giving 0.49 g (72%) (2S)-3-Methyl-2-{N-[2′-(1H-tetrazol-5-yl)-biphenyl-4-ylmethyl]-N′-(thiophene-2-carbonyl)-amino}-butanoic acid

Method D: The reaction was done according to the method C. N,N-dimethylformamide or N-methyl-pyrrolidone were used instead of acetonitrile.

### (2S)-3-Methyl-2-({-[2-(1H-tetrazol-5-yl)-biphenyl-4-ylmethyl]-N′-(thiophene-2-carbonyl)-amino}-butanoic acid (10)

(1S)—N-(1-Hydroxymethyl-2-methyl-propyl)-N′-[2′-(1H-tetrazol-5-yl)-biphenyl-4-ylmethyl]-thiophene-2-carboxamide (9) (5 g, 11.2 mmol) was dissolved in dichloromethane (60 ml) and 18crown6 (0.6 g, 2.3 mmol) was added. KMnO₄ (1.8 g, 11.4 mmol) dissolved in 0.5M NaOH (6 ml) was added drop-wise within 2 hours, and then mixture was stirred for 10 hours at laboratory temperature. Then Na₂SO₃ (20.0 g) and 35-% HCl (16 ml) were added and reaction mixture was stirred until black suspension of MnO₂ dissolve. Organic and aqueous phase were separated and organic layer was washed with 2M NaOH (100 ml) and water (100 ml). Combined alkaline and aqueous phase were extracted with dichloromethane (twice 100 ml), acidified with 35-% HCl to pH 2 and extracted with dichloromethane (twice 10 ml). Organic layer was dried over Na₂SO₄ and evaporated on RVE to yield 4.0 g (80.0%) of product. HR-MS: 462.1600 (MK', calc. for C₂₄H₂₄N₅O₃S⁺: 462.1617).

### (2S)-3-Methyl-2-{N-pentanoyl-N′-[2′-(1H-tetrazol-5-yl)-biphenyl-4-ylmethyl]-amino}-butanoic acid (Valsartan)

(2S)-3-Methyl-2-{N-[2′-(1H-tetrazol-5-yl)-biphenyl-4-ylmethyl]-N′-(thiophene-2-carbonyl)-amino}-butanoic acid (VS.INT7) (0.4 g, 0.87 mmol) dissolved in methanol (5 ml) and Raney Nickel was added. Reaction mixture was stirred in hydrogen atmosphere at normal pressure at room temperature for 24 hours. Reaction mixture was filtered through celite and evaporated on RVE to yield 0.35 g (90%) of Valsartan. HR-MS: 436.2349 (MH⁺, calc. for C₂₄H₃₀N₁₅O₃⁺: 436.2353).

1. A method for the production of a compound of general formula I 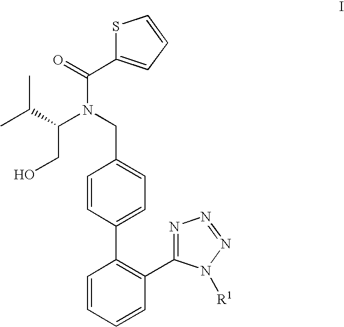
wherein R¹ represents hydrogen or a tetrazole protecting group, said method comprising the steps of:
reacting a compound of general formula II 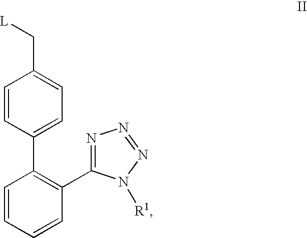
wherein R¹ represents hydrogen or a tetrazole protecting group and L represents a leaving group, with a compound of the formula III or a pharmaceutically acceptable salt thereof 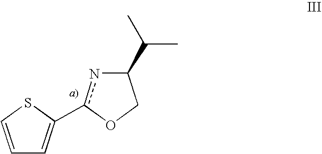
wherein a) denotes a double bond or a single bond, further wherein when a) denotes a single bond, the nitrogen atom is additionally substituted by a hydrogen atom.

2. The method according to claim 1, wherein said reaction step is carried out in the presence of a Bronsted base.

3. The method according to claim 1, wherein the compound of formula III wherein a) denotes a single bond and the nitrogen atom is additionally substituted by a hydrogen atom is used and wherein the reaction intermediate (4S)-3-[2′-(1-triphenylmethyl-1H-tetrazole-5-yl)-biphenyl-4-yl-methyl]-4-isopropyl-2-(2-thienyl)-1,3-oxazolidine is optionally isolated, optionally converted to (4S)-3-[2′-(1H-tetrazole-5-yl)-biphenyl-4-yl-methyl]-4-isopropyl-2-(2-thienyl)-1,3-oxazolidine, and further converted to I.

4. The method according to claim 1, wherein the compound of formula III wherein a) denotes a single bond and the nitrogen atom is additionally substituted by a hydrogen atom is obtained by the reaction of L-valinol with thipophene-2-carbaldehyde.

5. The method according to claim 1, wherein the compound of formula III wherein a) denotes a double bond is obtained by cyclizising a compound of formula IV

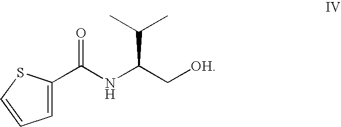

6. The method according to claim 5, wherein the compound of formula IV is obtained by reducing a compound of general formula V 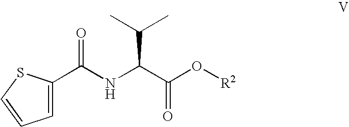
wherein R² represents alkyl or benzyl.

7. The method according to claim 6, wherein the compound of general formula V is obtained by reacting a compound of formula VI 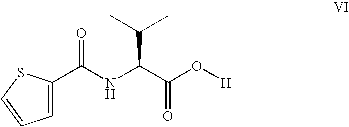
with a chlorinating agent and R²—OH wherein R² is defined as in claim 6.

8. The method according to claim 7, wherein the compound of formula VI is obtained by reacting a compound of general formula VII 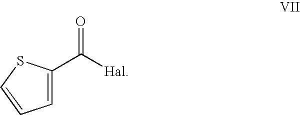
wherein Hal represents halogen,
with a compound of formula VIII

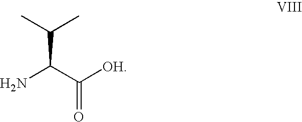

9. The method according to claim 1, wherein the compound of formula I comprises valsartan.

10. The method of claim 9, further comprising the step of oxidizing a compound of general formula I 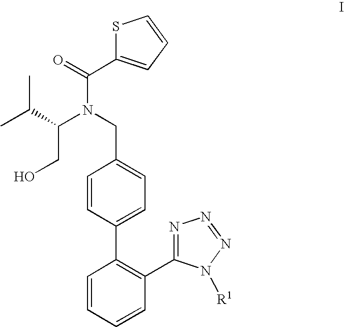
wherein R¹ represents hydrogen or a tetrazole protecting group, with either a compound of general formula IX 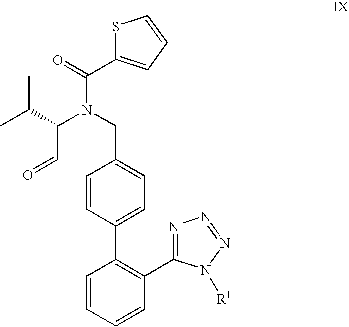
wherein R¹ represents hydrogen or a tetrazole protecting group, or with a compound of general formula X 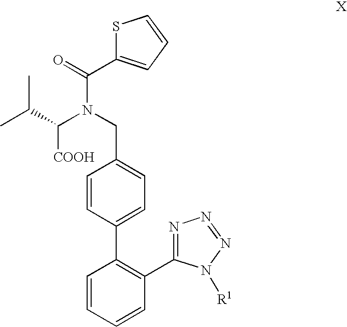
wherein R¹ represents hydrogen or a tetrazole protecting group.

11. A compound of the formula VI

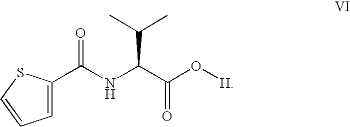

12. A compound of the general formula V 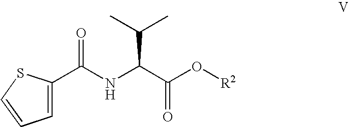
wherein R² represents alkyl or benzyl.

13. A compound of the formula IV

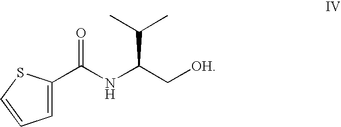

14. A compound the formula IIIa

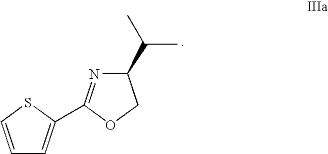

15. A compound of the formula IIIb

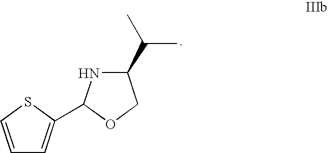

16. A compound comprising (4S)-3-[2′-(1-triphenylmethyl-1H-tetrazole-5-yl)-biphenyl-4-yl-methyl]-4-isopropyl-2-(2-thienyl)-1,3-oxazolidine having the formula

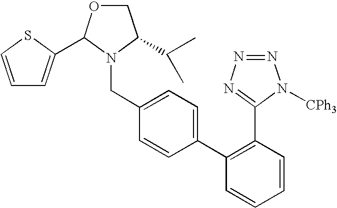

17. A compound comprising (4S)-3-[2′-(1H-tetrazole-5-yl)-biphenyl-4-yl-methyl]-4-isopropyl-2-(2-thienyl)-1,3-oxazolidine having the formula

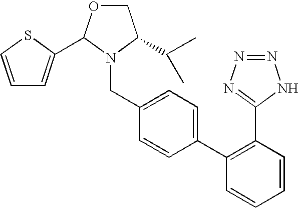

18. A compound of general formula I 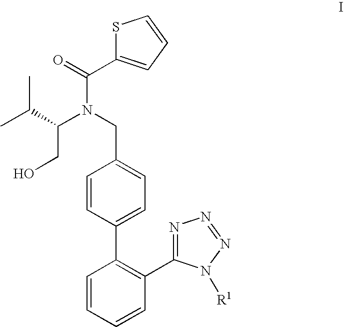
wherein R¹ represents hydrogen or a tetrazole protecting group.

19. A compound of general formula IX 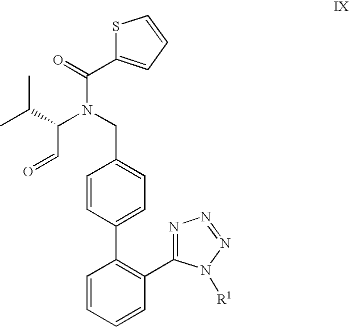
wherein R¹ represents hydrogen or a tetrazole protecting group.

20. A compound of general formula X 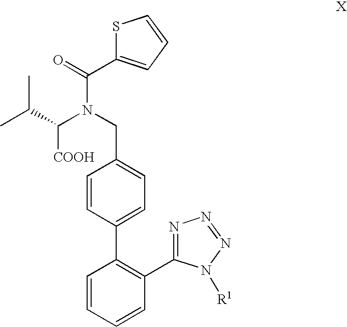
wherein R¹ represents hydrogen or a tetrazole protecting group.

21. (canceled)
